(19) United States
(12) Patent Application Publication
(10) Pub. No.: US 2023/0172481 A1
(43) Pub. Date: Jun. 8, 2023

(54) IMPLANTABLE CARDIOVASULAR MONITORING SYSTEM

(71) Applicants: Thomas Jefferson University, Philadelphia, PA (US); RTM Vital Signs, LLC, Philadelphia, PA (US)

(72) Inventors: Jeffrey I Joseph, Penn Valley, PA (US); Josiah Verkaik, Boise, ID (US)

(21) Appl. No.: 18/071,858

(22) Filed: Nov. 30, 2022

Related U.S. Application Data
(60) Provisional application No. 63/285,149, filed on Dec. 2, 2021.

(51) Int. Cl. A61B 5/07; A61B 5/0215; A61B 5/00

(52) U.S. Cl. CPC A61B 5/076; A61B 5/0215; A61B 5/6882; A61B 5/6876; A61B 2562/0247

(57) ABSTRACT

A sensor module retaining device having a first portion, a second portion, and a third portion. The first portion defines an open loop that is sized and configured to contour around an outer surface of a blood vessel. The second portion is opposite the third portion and the second and third portions each extend distally away from the open loop and define an open channel therebetween. The second and third portions are contiguous with and converge inwards towards the first portion.

### CROSS-REFERENCE TO RELATED APPLICATION

This application claims the benefit of U.S. Application Ser. No. 63/285,149, filed Dec. 2, 2021.

### FIELD

This disclosure relates to a method and system for an implantable cardiovascular monitoring system.

### BACKGROUND

More than 1,000 outpatients die every day in the United States suddenly (0.1% of the U.S. population each year). In August 2020, the National Center for Biotechnology Information (NCBI) estimated that up to 80% of sudden deaths outside of the hospital were related to cardiovascular disease. Essentially, the vital signs of these victims were not monitored in real-time or recorded for later analysis.

Recent advances in microelectromechanical systems (MEMS) sensors, electronics, flash memory, rechargeable battery power, telemetry, software, and data analytics have led to an increased interest in the development and commercialization of a long-term implantable Cardiovascular Monitoring System (CMS) with clinical accuracy, particularly for those at highest risk for an adverse event due to uncontrolled hypertension, myocardial ischemia, myocardial infarction, heart failure, diabetes, and other chronic cardiovascular diseases.

Cardiovascular disease is the leading cause of morbidity and mortality world-wide. Hypertension, also known as high blood pressure (BP), is the number-one risk factor for fatal strokes, fatal heart attacks, congestive heart failure, chronic kidney failure, dementia, and sudden death. Nearly half of all adults in the United States have hypertension, while only about 25% have their disease under satisfactory control. Approximately 13 million people in the United States have resistant hypertension, defined as uncontrolled hypertension despite treatment with diet, exercise, and three or more anti-hypertensive medications. Despite better medications for treating hypertension, poorly controlled blood pressure remains the major cause of premature death in adults making it responsible for every 1 in 8 deaths. More timely and effective diagnosis and treatment of chronic cardiovascular disease, especially uncontrolled hypertension, heart failure, myocardial ischemia, and valve disease, will likely cause a significant decrease in the incidence and severity of serious adverse cardiovascular events.

Arteriolar sclerosis and atherosclerosis are often cited by researchers as the mechanism by which chronic hypertension causes artery and arteriole stiffening, and chronic/acute arterial blood flow obstruction (myocardial infarction, stroke, peripheral vascular disease). In addition, the increased peripheral vascular resistance caused by chronic hypertension leads to left ventricular hypertrophy, ischemic heart disease, systolic/diastolic heart failure, and aortic/mitral valve insufficiency.

The measurement of BP using an upper arm blood pressure cuff, is routinely done in a doctor's office or clinic using manual or oscillometric techniques. Infrequent clinic measurements of the systolic, mean, and diastolic blood pressure at rest remain the most common method for diagnosing and managing patients with hypertension.

Systemic arterial BP is routinely measured for clinical care using an upper arm cuff device with the patient sitting still with their feet on the floor for at least 5 minutes. However, serious cardiovascular adverse events typically occur during and after exercise and stressful events. Dynamic changes in arterial BP have been identified during ambulatory BP monitoring in healthy and hypertensive patients: 1) normal 20% to 30% decrease in BP during sleep, 2) normal 20% to 30% increase in BP upon awakening, 3) normal>20% increase in BP during daily activities, 4)>30% increase in BP during exercise, and 5) a lack of BP decrease during sleep and a persistent increase in BP during daily activities in patients with uncontrolled hypertension. Many patients experience a significant decrease in BP with symptoms due to medications, dehydration, and upon moving from the supine to the standing position (orthostatic hypotension). Even a brief period of hypotension can cause dizziness, trauma due to a fall, and end-organ damage of the brain, heart, and kidneys.

Automated oscillometric devices are now available that use an upper arm cuff to measure the systolic, mean, and diastolic BP of ambulatory patients every 30 to 60 minutes for 24 to 48 hours. Ambulatory BP monitoring has gained clinical acceptance to diagnose and manage hypertension, especially in patients that may have “white coat” hypertension; an elevated BP that resolves once outside of the physician's office. The use of ambulatory BP monitoring is time-limited due to the discomfort of cuff inflation and the inconvenience of wearing a bulky device. However, an ambulatory patient's arterial BP fluctuates constantly in response to the physiological stimuli of everyday living, and as such, the intermittent measurement of the arterial BP at rest as the most important risk factor for cardiovascular disease appears inconsistent with a large body of data on hypertension. The clinical focus on an elevated mean or systolic BP measurement is arguably questionable, because an elevated diastolic pressure, a variable BP, and an increased pulse pressure have all been associated with an increased risk for a serious cardiovascular adverse event.

Recently, there has been an increased interest in the importance of BP variability rather than mean or systolic BP in predicting the risk for cardiovascular disease. Re-analysis of large clinical trials has shown that the variability between in-office BP measurements is a predictor of cardiovascular events, independent of the mean BP. The measurement devices used in these trials provided few data points at rest and were unable to measure the dynamic and transient changes in BP that occur during daily activities.

Physicians frequently recommend that patients self-monitor their BP at home using an oscillometric device with an upper arm cuff, or an inflatable cuff around the wrist. Unfortunately, an accurate measurement requires the patient to remain still with the cuff held at the heart level for more than 60 seconds. Modern devices can communicate wirelessly with a central monitoring station and electronic medical record with computer data analytics to enhance the clinical efficacy of remote patient monitoring.

Existing non-invasive wearable devices (patches and smart watches) utilize pulse transit time to estimate systolic arterial blood pressure. The devices measure the electrocardiogram to detect the onset of systole and measure the onset of the peripheral pulse wave using photoplethysmography. The measured pulse transit time correlates with the systolic arterial blood pressure, but is also influenced by arterial elasticity or compliance. The technology of pulse transit time is flawed because the slope of the relationship with arterial BP differs between individuals and in the same individual during physiological perturbations, and is very sensitive to motion artifact. Additionally, known wearable monitors using pulse transit time require patients to remain still for 60 seconds during a measurement, frequent calibration with an upper arm cuff, and are not accurate enough to diagnose hypertension or dose medication.

Physicians in the hospital routinely insert a vascular catheter into the lumen of a radial, brachial, or femoral artery to continuously monitor the arterial blood pressure waveform. The catheter is attached to fluid filled tubing and an external pressure transducer hard-wire connected to a bedside data acquisition system and display. Invasive arterial blood pressure monitoring has been performed in the emergency rooms, operating rooms, and intensive care units of hospitals for more than 50 years to enhance the timeliness and accuracy of diagnosis, with the goal of preventing a serious adverse event. Recent advances in arterial waveform analysis provide diagnostic information for calculating important heart function parameters beyond systolic, mean, and diastolic BP, and is more accurate than intermittent BP cuff measurements.

A few clinical trials in ambulatory outpatients with hypertension have used a brachial arterial catheter, external pressure transducer, and a data logger to record the dynamic changes in the arterial blood pressure waveform over a 24 to 48 hour period. The risk of arterial bleeding and infection limit this method to short-term monitoring of highly compliant patients.

### SUMMARY

The present invention provides a method and system for an implantable cardiovascular monitoring system. The device is a sensor module retaining device that has a first portion defining an open loop sized and configured to contour around an outer surface of a blood vessel. The sensor module retaining device also has a second portion and a third portion opposite the second portion, the second and third portions extending distally away from the open loop and defining an open channel therebetween, the second and third portions being contiguous with and converging inwards towards the first portion.

In another aspect of this embodiment, the first portion has a first end and a second end opposite the first end, the first end being contiguous with the second portion and the second end being contiguous with the third portion.

In another aspect of this embodiment, the open loop is defined between the first and second ends.

In another aspect of this embodiment, the first portion defines a plurality of apertures to promote tissue ingrowth.

In another aspect of this embodiment, the second and third portions each define at least one opening.

In another aspect of this embodiment, the first portion defines an open lumen and the open channel has a width larger than a width of the open lumen.

In another aspect of this embodiment, the plurality of apertures are uniformly spaced about the first portion.

In another aspect of this embodiment, the sensor module retaining device is made of at least one substantially rigid material selected from the group consisting of titanium, stainless steel, MP35N alloy, plastic, and ceramic.

In another aspect of this embodiment, at least a portion of the first portion is textured to facilitate adhesion of the sensor module retaining device to the outer surface of the blood vessel.

In another aspect of this embodiment, the open channel is sized to receive a sensor module having one of: a width equal to a width of the blood vessel; and a width larger than a width of the blood vessel.

In another embodiment, a system for measuring vital signs of a patient having a blood vessel, comprises a sensor module having a housing, the sensor module being configured to measure a blood vessel blood pressure waveform, and a sensor module retaining device releasably coupled to the sensor module. The sensor module retaining device includes a first portion defining an open loop sized and configured to contour around an outer surface of the blood vessel a second portion and a third portion opposite the second portion, the second and third portions extending distally away from the open loop and defining an open channel therebetween, the second and third portions being contiguous with and converging inwards towards the first portion.

In another aspect of this embodiment, the sensor module includes a diaphragm coupled to the housing and wherein the sensor module is received within the open channel such that diaphragm is in contact with the outer surface of the blood vessel.

In another aspect of this embodiment, at least the first portion of the sensor module retaining device defines a plurality of apertures to promote tissue ingrowth.

In another aspect of this embodiment, the first and second portions each define at least one opening sized and configured to engage at least one corresponding mating member located on the sensor module.

In another aspect of this embodiment, the second and third portions each have a proximal segment and a distal segment contiguous with the proximal segment, the proximal segment of the second and third portions being contiguous with the first portion.

In another aspect of this embodiment, the open loop is defined between the first and second ends.

In another aspect of this embodiment, the first portion defines an open lumen and the open channel has a width larger than a width of the open lumen.

In another aspect of this embodiment, the plurality of apertures are uniformly spaced about the first portion.

In another aspect of this embodiment, at least a portion of the first portion is textured to facilitate adhesion of the sensor module retaining device to the exterior surface of the blood vessel.

In another embodiment, a system for measuring vital signs of a patient having a blood vessel, comprises a sensor being configured to measure a blood vessel blood pressure waveform. The sensor includes a sensor module having a housing and a diaphragm, the diaphragm being coupled to the housing. The sensor module retaining device is releasably coupled to the sensor module. The sensor module retaining device includes a first portion defining an open loop sized and configured to contour around an outer surface of the blood vessel, the first portion of the sensor module retaining device further defining a plurality of apertures to promote tissue ingrowth and a second portion and a third portion opposite the second portion, the second and third portions extending distally away from the open loop and defining an open channel therebetween, the second and third portions being contiguous with and converging inwards towards the first portion, and when the sensor module is received within the housing the diaphragm is in contact with the outer surface of the blood vessel.

### BRIEF DESCRIPTION OF THE DRAWINGS

A more complete understanding of embodiments described herein, and the attendant advantages and features thereof, will be more readily understood by reference to the following detailed description when considered in conjunction with the accompanying drawings wherein:

FIG. 1 is a perspective view of an embodiment of an implantable cardiovascular monitoring system constructed in accordance with the principles of the present application and a view of a sensor module and sensor module retaining device coupled to a patient's blood vessel;

FIG. 2 is a perspective view showing the sensor module coupled to sensor module retaining device;

FIG. 3 is a cross-sectional view showing how the sensor module retaining device of FIG. 1 is sized and configured to contour around an outer surface of the patient's blood vessel, and how the coupling of the sensor module retaining device and the sensor module results in applanation of the blood vessel;

FIG. 4 is a perspective view of the sensor module retaining device of FIGS. 1-3; and

FIG. 5 is a cross-sectional view showing the coupling of the sensor module and the sensor module retaining device.

### DETAILED DESCRIPTION

Before describing in detail exemplary embodiments, it is noted that the embodiments reside primarily in combinations of apparatus components and processing steps related to an implantable cardiovascular monitoring system. Accordingly, the system and method components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

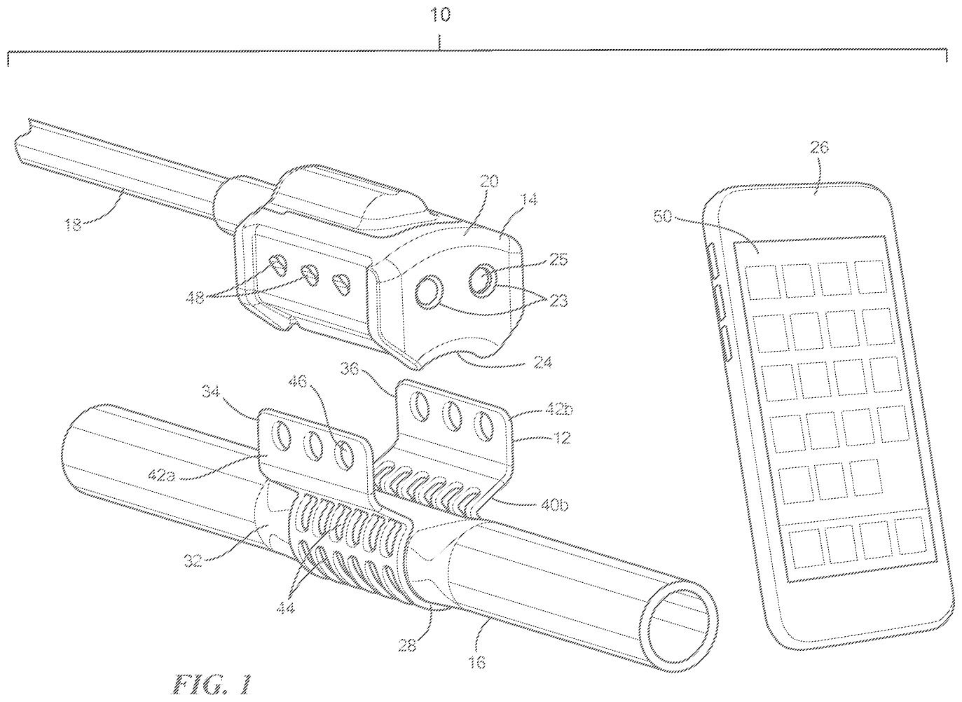

Referring now to FIGS. 1-5, is an implantable Cardiovascular Monitoring System 10 for measuring vital signs of a patient having a blood vessel is shown. The implantable Cardiovascular Monitoring System (CMS) 10 will overcome the limitations of traditional BP measurement devices by continuously measuring and analyzing the detailed arterial BP waveform with real-time alerts and alarms based upon the vital sign trend data. The arterial BP waveform can be analyzed to determine a patient's heart rate, stroke volume, cardiac output, myocardial contractility, systemic vascular resistance, systolic/diastolic timing intervals, and valve function-important metrics for diagnosing and treating chronic cardiovascular disease.

Diagnostic algorithms within the CMS 10 will detect subtle and overt changes in the normal pattern of the individual patient's trend data, hours and days prior to a serious cardiovascular adverse event. CMS 10 data can be transmitted via the cellular network to a central monitoring station for advanced analysis by a computer and clinician, with near real-time feedback to the patient and summary trend data to the primary care physician.

In one embodiment, the implantable CMS 10 may continuously measure and analyze the patient's core body temperature, electrocardiogram, hemoglobin oxygen saturation, arterial photoplethysmograph, respiratory rate, tidal volume, minute ventilation, breathing pattern, number/duration of apnea events, sounds of the upper airway, heart, and lungs, body position, and activity level.

As shown in FIG. 1, the system 10 includes a sensor module retaining device 12, sized and configured to receive, retain, and removably couple a sensor module 14 to an artery or target blood vessel 16 within the body of a patient. Upon implantation, the sensor module retaining device 12 may surround select portions of the blood vessel 16 without necessitating the complete encircling of the blood vessel 16. The sensor module 14 may be an arterial applanation tonometer fully or partially implanted within the patient and having at least one sensor (as described in more detail below) configured to measure a blood pressure waveform from the target blood vessel 16 and is connected to an implantable electronic module or electronics communication assembly (not shown) via a flexible electric lead 18. However, in other configurations, in addition to continuously measuring a patient's blood pressure waveform, the sensor module 14 may also be configured to continuously measure and analyze the patient's electrocardiogram, hemoglobin oxygen saturation, photo-plethysmograph waveform (pulse oximeter), temperature, respiratory rate, tidal volume, breathing pattern, body position, activity level, and sounds of the upper airway, lungs, and heart (phonocardiogram). Although not shown, it is to be understood that the electronics module can generally contain a motherboard with integrated circuit (IC) chips, control software, diagnostic software, flash memory, an antennae, and a rechargeable battery.

The sensor module 14 may be made from a biocompatible, non-degradable, and rigid material such as titanium, MP35N alloy, plastic, or ceramic having a low density, relatively high strength, and a relatively high level of corrosion resistance. When coupled together, the sensor module 14 and sensor module retaining device 12 form a fixed (non-compliant) interface or structure. The sensor module 14 may be implanted around the outer surface of any peripheral artery or target blood vessel (i.e., peripheral artery, internal thoracic/mammary artery, superior thoracic artery, thoracoacromial artery, thoracic-dorsal artery, lateral thoracic artery, external carotid artery, and the superior epigastric artery, etc.) using manual surgical methods and/or a customized surgical tool configured for positioning and attachment of the sensor module to the target blood vessel. An electronics module (not shown) may then be implanted within the subcutaneous tissue of the chest wall of the patient. The sensor module 14 may also be implanted using local anesthesia with sedation and aseptic surgical technique. The clinician may use external ultrasound to image the outer diameter of the target artery (i.e., the target blood vessel 16) prior to surgical implantation. Based upon the ultrasound measurements, the surgeon may choose an inner diameter of the sensor module retaining device 12 to produce an amount of applanation (10% to 25% flattening) of the target blood vessel 16. Using the ultrasound measurements to select the inner diameter of the sensor module retaining device 12 may assist in producing an optimal amount of applanation for a particular patient. The sensor module 14 can be implanted around the outside of the target blood vessel 16 manually or using a J-shaped surgical tool. Implanting the sensor module 14 around the outside of a peripheral artery at the level of the aortic valve will minimize the effect of a change in body position (gravity) on the BP measurement. Alternatively, the sensor module 14 may be created in a variety of different widths and the width of the sensor module 14 may be selected to produce an optimal amount of applanation for a patient.

Although not described in detail herein for simplicity, it is to be understood that the sensor module 14 generally includes a housing 20 sized and configured to retain at least one pressure sensor 21, temperature thermistor, silicone fluid filled reservoir, and processing circuitry (not shown), and a diaphragm 22 coupled to the inferior surface 24 of the housing 20. In one exemplary configuration, for example, as shown in FIG. 1, the housing 20 may have a width larger than a width or diameter of the target blood vessel 16. However, in other configurations, the housing 20 may also have a width equal to or smaller than the width or diameter of the target blood vessel 16.

Surgical implantation of the arterial applanation tonometer sensor head and sensor module retaining device 12 can be accomplished using a manual method or a surgical tool. The coupling of the sensor module's 14 diaphragm 22 to the outside wall of the target blood vessel 16 in a region of applanation or flattening can impact long-term performance. The sensor module retaining device 12 must maintain mechanical coupling of the diaphragm 22 within the flattened region of target blood vessel 16 over the physiological range of vasodilation and vasoconstriction. For example, a 2.5 mm outer diameter peripheral artery may increase 700 micrometers during systole and decreased 700 um in diameter during diastole. The target blood vessel 16 can increase up to 20% in outer diameter due to a moderate increase in systolic/diastolic/mean blood pressure and decrease up to 50% in outer diameter due to vasoconstriction (spasm) or very low blood pressure. A significant decrease in outer diameter may adversely affect mechanical coupling of the diaphragm 22 to the outer wall of the target blood vessel 16.

The sensor module retaining device 12 defines openings or apertures (discussed below) that facilitate the ingrowth of adjacent connective tissue and artery wall tissue that can enhance mechanical coupling of the sensor module retaining device 12 with vessel wall tissue. In addition, the inferior surface 24 of the sensor module 14 can also have a textured or porous architecture designed to facilitate further ingrowth of adjacent arterial wall tissue. Tissue that grows through the openings or apertures of the sensor module retaining device 12, and grows into the sensor module's 14 textured inferior surface 24, mechanically secures or couples the diaphragm 22 to the wall tissue of the target blood vessel 16 in the region of applanation. This particular configuration produces a strong and robust coupling of the diaphragm 22 to the wall of the target blood vessel 16 despite vasocontriction and eliminates body motion induced artifact or noise. The ingrowth of adjacent connective tissue also enhances the mechanical strength of the wall tissue of the target blood vessel (eliminates thinning) and blood supply of the target blood vessel (vaso vasorum).

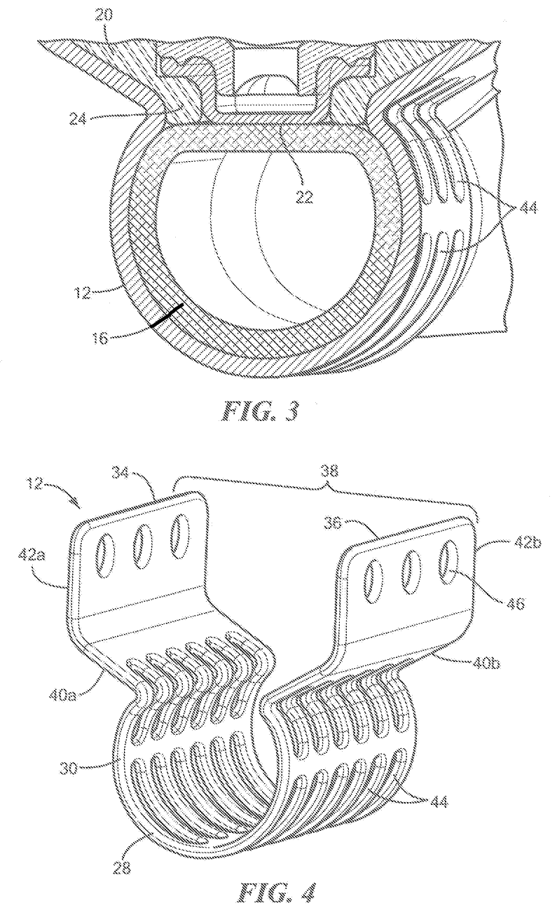

As shown in FIG. 3, when coupled to the sensor module retaining device 12, the sensor module 14 is positioned such that the diaphragm 22 is in contact with the outer surface of the blood vessel 16, which allows pressure sensor 21 disposed within the sensor module 14 to measure a change in the hydraulic pressure when a force is imparted on the diaphragm 22 by the blood vessel 16. In other words, pressure sensor 21 is configured to measure at least one blood pressure waveform from the target blood vessel 16. Once the blood pressure waveform of the blood vessel 16 is measured and recorded, the processing circuitry of the sensor module transmits the recorded measurements to a remote control unit 26 (discussed in more detail below) via a low-energy Bluetooth® signal or other wireless protocol such as the Medical Device Radiocommunication Service (MICS).

Although not shown in detail herein, the housing 20 may define a fluid chamber sealed by the diaphragm 22. A fluid, such as silicone fluid, may be disposed within the fluid chamber for exhibiting a hydraulic pressure consistent with the pressure applied to the diaphragm 22. Hydraulic generally refers to fluid in a confined space (closed system) wherein the fluid is a medium to transmit force. This is in accordance with the discovery of Pascal that a pressure applied to any part of a confided fluid transmits to every other part with no loss. The pressure acts with equal force on all equal areas of the confining walls in a direction perpendicular to the wall surfaces. The pressure sensor 21 of the sensor module 14 is configured to measure a change in the hydraulic pressure when the force is imparted on the diaphragm 22, such as on the inferior surface 24, or any other outer surface, of the diaphragm 22 by the blood vessel 16. As such, the diaphragm 22 may be a compliant diaphragm configured to be secured, i.e., mechanically coupled, against a wall of the blood vessel 16 to facilitate transduction of the blood pressure through a wall of the blood vessel 16. The pulsatile intravascular blood pressure is transmitted across the flatten artery wall tissue and across the diaphragm 22 into the silicone fluid filled reservoir without distortion or loss of energy. The MEMS pressure die measures the pulsatile pressure within the reservoir with satisfactory accuracy and detail for diagnosing hypertension, hypotension, heart failure, myocardial ischemia, valve dysfunction, etc., and dosing medication.

In some configuration, the hydraulic pressure within the diaphragm 22 can be measured by the pressure sensor 21 of the sensor module 14, with the pressure sensor 21 being a resistive or capacitive sensor, for example, a MEMS sensor or MEMS pressure die. As a non-limiting example, the pressure sensor 21 may be that which is marketed and sold under the name NovaSensor® or the pressure sensor 21 may be a silicon, micro-machined, piezo resistive pressure sensing chip within the Smi510E Series, such as, for example SM5108E, owned by Silicon Microstructures, Inc. In other configurations, the pressure sensor 21 may be another type of sensor configured to measure hydraulic pressure. In an alternate configuration, the inner surface of the sensor module 14 can be made from a single piezoelectric force transducer or an array of force transducers (for example 2, 4, 6, 8, 16, 32, 64, 128, 256 force transducers on a 1 mm×6 mm computer chip) coupled to the outer artery wall of the target blood vessel 16 within a region of applanation.

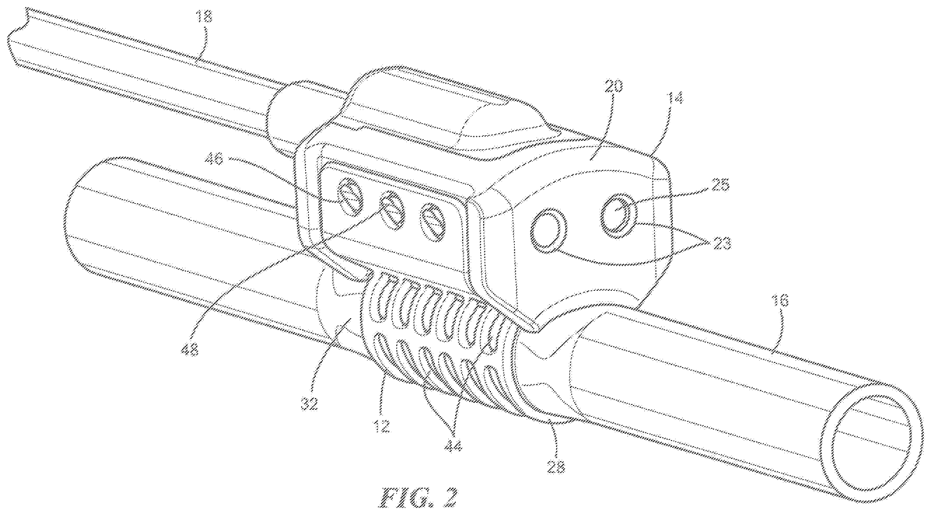

As shown in FIGS. 1 and 2, in order to insert the fluid within the fluid chamber, the housing 20 defines one or more ports 23, for example a fill port and a vent port, in fluid communication with the fluid chamber. The fill port may be used to deposit and adjust the amount of the fluid, e.g., silicone, located within the fluid chamber, whereas the vent portion may be used to receive a vacuum to remove any entrapped air from the fluid chamber. The removal of air assists in providing the fluid that is free of air bubbles in an effort to obtain accurate hydraulic pressure measurements. As shown in FIGS. 1 and 2, a stopper 25, for example a ceramic ball, may be disposed within each of the ports 23 to provide a hermetic seal.

Referring now to FIG. 3, in some configurations, the sensor module retaining device 12 may be sized and configured to minimize the change in lumen shape and cross-section area of the target blood vessel 16. The sensor module 14 may be connected to and disconnected from the superior arms (e.g., the second and third portions 34, 36, discussed below) of the sensor module retaining device 12. The inner diameter of the sensor module retaining device 12 may impact the amount or degree of applanation. The artery wall tissue of the target blood vessel 16 will remodel to take on the D-shape of the lumen produced by the connected sensor module 14 and sensor module retaining device 12. The sensor module 14 can be manufactured with one size (for example 8 mm length×1.6 mm width×8 mm height), while the sensor module retaining device 12 can be made with a wide range of inner diameter sizes to accommodate an artery with an outer diameter 1.8 mm to 3.2 mm (manufactured 200 μm larger or smaller diameter). The sensor module 14 can be manufactured with a range of widths (as non-limiting examples the widths may be 1.2 mm, 1.4 mm, 1.6 mm. 1.8 mm, and 2.0 mm) to accommodate a range of artery outer diameters, because the artery diameter influences the area of artery wall tissue located in the region of applanation. The 1.2 mm width of the inferior surface 24 of the sensor module 14 is the minimum amount of applanation because the 1 mm width diaphragm needs to be located within the region of artery wall tissue flattening. A sensor module 14 with a smaller width could be used for a smaller artery and a larger width could be used for a larger artery.

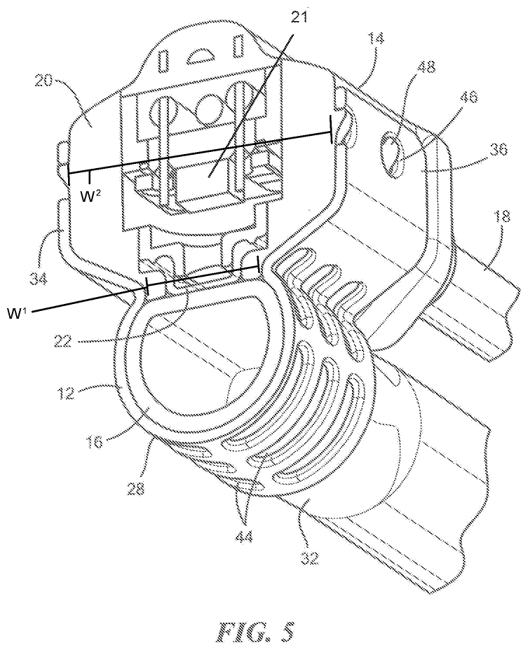

Continuing to refer to FIGS. 3 and 4, an exemplary sensor module retaining device 12 is shown that is made out of a biocompatible, non-degradable, and rigid material such as titanium, stainless steel, MP35N alloy, plastic, or ceramic with, relatively high strength, and corrosion resistance. The sensor module retaining device 12 includes a first portion 28 that defines an open loop 30 sized and configured to contour around an outer surface 32 of the target blood vessel 16. In other words, the open loop 30 is defined by a curvature of the first portion 28 such that at least a portion of the first portion 28 may be flush with and curve along the outer surface 32 of the target blood vessel 16. In one embodiment, the open loop 30 defined by the first portion 28 may have a diameter sufficiently large to receive and couple to a target blood vessel having an outer diameter between at least 1.8 mm to 3.2 mm. The sensor module retaining device 12 also includes a second portion 34 and a third portion 36 opposite the second portion 34. As shown in FIG. 4, the second and third portions 34, 36, are each contiguous with and converge or taper inwards towards the first portion 28. The open loop 30 is defined by the first portion 28 between second and third portions 34, 36. Also, the second and third portions 34, 36, each extend distally away from the open loop 30 and are sized and configured to define an open channel 38 therebetween. As shown in FIGS. 1, 4, and 5, the defined open channel 38 is sized to receive the sensor module 14. In embodiments wherein the inferior surface 24 of the sensor module 14 has a width larger than the width of the target blood vessel 16, the open channel 38 may also have a width larger than the width of the target blood vessel 16. As such, the open channel 38 may therefore also have a width larger than a width of the open loop 30. Further, as described herein, it is to be understood that the first, second, and third portions 28, 34, 36 of the sensor module retaining device 12 may each have rounded or curved edges to minimize the potential for sharp edges to be in contact with soft tissue of a target blood vessel or other surrounding tissue structures.

Continuing to refer to FIG. 4, the second and third portions 34, 36, may each include multiple contiguous segments. For example, the second and third portions 34, 36, may each include a proximal segment 40a, 40b, and a distal segment 42a, 42b, respectively. The distal segments 42a, 42b, are substantially orthogonal or angled in relation to each respective proximal segment 40a, 40b. The proximal segment 40a, 40b of each portion 34, 36, is proximate to the first portion 28. The distal segments 42a, 42b of each portion 34, 36, are distal to and extend distally away from the first portion 28. The proximal segments 40a, 40b each converge or taper inwards from the distal segments 42a, 42b towards the first portion 28.

In some configurations, the sensor module retaining device 12 may be substantially U-shaped such that it may contour around the perimeter of the target blood vessel 16. The sensor module retaining device 12 and the sensor module 14 can be pre-loaded on a substantially J-shaped surgical tool (not shown). When implanting the device 12, the clinician can isolate a 10 mm length of the target blood vessel 16 and remove the outer tunica adventitia. The target blood vessel 16 can be advanced through the gap located between the inferior surface 24 of the sensor module 14 and the sensor module retaining device 12. The target blood vessel 16 becomes located within the sensor module retaining device 12. The tool can be “fired” to connect the sides of the sensor module 14 to the section and third portions 34, 36 of the sensor module retaining device 12 in the optimal x-y-z alignment and degree of applanation. The implantation tool can then be removed from the surgical site and the sensor head's lead attached to an implantable electronics module.

Now referring to FIGS. 1, 4, and 5, the entirety of the sensor module retaining device 12, or at least the first portion 28, may be textured, surface modified, or scaffold-like to facilitate the adhesion and ingrowth of wall tissue of the target blood vessel 16 and adjacent connective tissue. Also, the first portion 28 may define a plurality of openings or apertures 44. The plurality of apertures 44 may be arranged uniformly (as shown in FIG. 1) or in any other arrangement or pattern along the curvature of the first portion 28, and facilitate the ingrowth of adjacent tissue and vaso vasorum (blood supply) to the wall tissue of a target blood vessel 16. For example, when the sensor module retaining device 12 is coupled to the target blood vessel 16, tissue cells from the target blood vessel 16 may continue to grow in and between each aperture 44, thereby further binding the sensor module retaining device 12 to the target blood vessel 16. The tissue ingrowth will also enhance the strength, thickness, and health of the wall tissue. Similarly, the inferior surface 24 of the housing 20 may be textured, surface modified, or scaffold-like to facilitate the adhesion and ingrowth of wall tissue of a target blood vessel. Additionally, in one embodiment, at least a portion of the sensor module retaining device 12 or sensor module 14 may be coated with chemicals, medications, or other types of therapeutic agents or drugs that promote the ingrowth and adhesion of wall tissue and/or minimize acute and chronic inflammation. The adhesion or ingrowth of the wall tissue of a target blood vessel to the surface of the sensor module 14 enhances the mechanical coupling of the sensor module's 14 diaphragm 22 to the outer wall or surface 32 of the blood vessel 16 especially when the blood vessel diameter becomes smaller due to vasoconstriction or low blood pressure. In other words, the desired effect of the tissue adhesion or ingrowth is reliable mechanical coupling of the sensor module diaphragm 22 to a target blood vessel, while producing the smallest amount of applanation.

As shown in FIGS. 1, 4, and 5, the distal segments 42a, 42b of the second and third portion 34, 36, may further define at least one opening 46 sized to engage or receive at least one corresponding mating member 48 on the housing 20 of the sensor module 14. Each corresponding mating member 48 may be a button, stem, rod, or other type of protrusion sized and configured to be received through an opening 46. In one embodiment, the sensor module 14 is coupled to the sensor module retaining device 12 via a snap-fit connection between the openings 46 and the corresponding mating members 48 on the housing 20.

Now referring to FIGS. 4 and 5, the sensor module 14 is received within the open channel 38 and coupled to the sensor module retaining device 12 via the mating of the openings 46 and the at least one corresponding mating members 48. When the sensor module 14 is coupled to the sensor module retaining device 12, the sensor module 14 is adapted to minimize stress imposed on the blood vessel 16 in undergoing a forced transition from a circular geometry to a constrained flattening of a portion of the blood vessel (as shown in FIGS. 3 and 5) for achieving applanation and accurate blood pressure transduction through the blood vessel wall to the diaphragm 22. The design of the second and third portions 34, 36 of the sensor module retaining device 12 enable the sensor module 14 to least partially surround the blood vessel 16 without overly constraining the blood vessel 16 when obtaining the blood pressure measurements or performing alternative medical diagnostics. When fully coupled to the sensor module retaining device 12, the inferior surface 24 of the sensor module 14 is in contact with the outer surface 32 of the target blood vessel 16 to produce 10% to 15% to 25% applanation of the blood vessel 16.

Shortly after sensor implantation around the target blood vessel 16, the vessel 16 wall's connective tissue architecture, smooth muscle cells, and endothelia cells will remodel to take on the exact shape of the inner lumen-defined as the sensor module's inferior surface 24 and the inner surface of the sensor module retaining device 12. Tissue remodeling will eliminate the radial and elastic forces that are present at the time of acute vessel wall flattening-allowing the intravascular pressure to the transferred across the diaphragm with the least amount of vessel wall flattening or applanation.

In one embodiment the inferior surface 24 of the sensor module 14 may have a fixed width of approximately 1.6 mm (diaphragm 22 has 1 mm width and the housing 20 has a 0.3 mm width on each side). The pulsatile intra-vascular pressure is transmitted across the wall tissue of the blood vessel 16 and diaphragm 22 into the silicone fluid reservoir with minimal distortion or attenuation. Additionally, a portion of the inferior surface 24 which is near the diaphragm 22, may have a first width W₁ that is smaller than the width of the target blood vessel 16. Having this size width W₁ for the diaphragm 22 allows the diaphragm 22 to have contact with a portion of the target blood vessel 16. While the width of the sensor module 14 may be uniform, the sensor module 14 may have a second width W₂ which is in one embodiment near where the pressure sensor 21 is located in the housing 20. The second width W₂ may be larger than the first width W₁ and in some embodiment the second width W₂ may be larger than the width of the target blood vessel 16. The inferior surface 24 of the sensor module 14 and sensor module retaining device 12 may be textured to facilitate adhesion of the blood vessel tissue to the sensor module 14. Although not shown, the inferior surface 24 of the sensor module 14 may also include a light source and detector of an implantable pulse oximeter to continuously measure the photoplethysmograph and the hemoglobin oxygen saturation carried in the patient's red blood cells. Mechanically coupling the light source and light detector of the pulse oximeter directly on the outer wall of the blood vessel produce an excellent signal to noise ratio and eliminates artifact due to body movement.

Continuing to refer to FIGS. 1 and 5, when fully coupled to the sensor module retaining device 12 and in contact with the target blood vessel 16, the sensor module 14 may continuously record the blood pressure waveform of the blood vessel 16. The measurements are then transmitted to the remote control unit 26 via the low-energy Bluetooth® signal. In some configurations, the remote control unit 26 can be connected to an external oscillometric BP monitor with an upper arm cuff. In some such configurations, the patient or clinical can intermittently calibrate the arterial BP waveform (systolic, mean, and diastolic BP) of the implanted sensor module 14 using one or more measurements made by the external oscillometric BP monitor. The calibration data can be transmitted to the sensor module 14 as frequently as necessary (for example every 7 to 28 days) to produce a BP measurement accurate enough to diagnose hypertension and dose medication. The non-compliant sensor module housing, non-complaint sensor module retaining device, and tissue ingrowth combine to produce a stable and secure mechanical coupling of the implanted sensor's diaphragm to the outside wall of the blood vessel. This stable interface minimizes sensor drift and the need for frequent calibration using an external oscillometric BP monitor. In other configurations, the remote control unit 26 may be an external control unit or computer operated by the clinician and/or the patient. In some configurations, the remote control unit 26 may also be a smart phone, smart watch, tablet, computer, or other type of smart device. Further, it is to be understood that in one embodiment, the sensor module 14 may be in communication with more than one remote control unit 26. For example, the sensor module 14 may be in communication with a patient's smart phone and an external control unit operated by a clinician. Once the blood pressure waveform signals are transmitted to the remote control unit 26, the processing circuitry of the remote control unit 26 may then analyze and display in real-time the recorded blood pressure measurements and also display any visual and/or audible alerts and alarms on a display or screen 50 of the remote control unit 26. The alerts and/or alarms may be indicative of an adverse event such as low, high, and/or abnormal blood pressure readings or patterns that are being observed or have been observed by the sensor module 14. As such, the alerts and/or alarms may also notify clinicians and/or patients as to whether the patient's levels of certain blood pressure regulating medications needs to be adjusted, such as to treat low, high, or abnormal blood pressure, or whether the patient needs to seek medical attention.

Once other vital sign sensor signals (electrocardiogram, hemoglobin oxygen saturation, arterial photoplethysmograph, core temperature, respiratory rate, tidal volume, minute ventilation, duration of apnea events, sounds of the upper airway, heart, and lungs, body position, and activity level are transmitted to the remote control unit 26, the processing circuitry of the remote control unit 26 may then analyze and display in real-time the recorded vital sign measurements and also display any visual and/or audible alerts and alarms on a display or screen 50 of the remote control unit 26. The diagnostic algorithms can detect subtle and overt changes in the normal pattern of an individual patient's vital sign trend data. The alerts and/or alarms may be produced when the algorithms detect or predict an increased risk for a serious adverse. As such, the alerts and/or alarms may notify clinicians and/or patients as to whether their cardiovascular medications need to be adjusted, or whether the patient needs to seek immediate medical attention. Vital sign trend data determined to be clinically important can be recorded into flash memory. Recorded vital sign trend data can be easily downloaded to an external controller or computer for review by a physician, nurse, or emergency personnel.

As used herein, relational terms, such as “first” and “second,” “top” and “bottom,” and the like, may be used solely to distinguish one entity or element from another entity or element without necessarily requiring or implying any physical or logical relationship or order between such entities or elements. The terminology used herein is for the purpose of describing particular embodiments only and is not intended to be limiting of the concepts described herein. As used herein, the singular forms “a”, “an” and “the” are intended to include the plural forms as well, unless the context clearly indicates otherwise. It will be further understood that the terms “comprises,” “comprising,” “includes” and/or “including” when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

In embodiments described herein, the joining term, “in communication with” and the like, may be used to indicate electrical or data communication, which may be accomplished by physical contact, induction, electromagnetic radiation, radio signaling, infrared signaling or optical signaling, for example. One having ordinary skill in the art will appreciate that multiple components may interoperate and modifications and variations are possible of achieving the electrical and data communication.

It will be appreciated by persons skilled in the art that the present embodiments are not limited to what has been particularly shown and described herein above. In addition, unless mention was made above to the contrary, it should be noted that all of the accompanying drawings are not to scale. A variety of modifications and variations are possible in light of the above teachings.

1. A sensor module retaining device, comprising:
a first portion defining an open loop sized and configured to contour around an outer surface of a blood vessel; and
a second portion and a third portion opposite the second portion, the second and third portions extending distally away from the open loop and defining an open channel therebetween, the second and third portions being contiguous with and converging inwards towards the first portion.

2. The device of claim 1, wherein the first portion has a first end and a second end opposite the first end, the first end being contiguous with the second portion and the second end being contiguous with the third portion.

3. The device of claim 2, wherein the open loop is defined between the first and second ends.

4. The device of claim 1, wherein the first portion defines a plurality of apertures to promote tissue ingrowth.

5. The device of claim 1, wherein the second and third portions each define at least one opening.

6. The device of claim 1, wherein the first portion defines an open lumen and the open channel has a width larger than a width of the open lumen.

7. The device of claim 4, wherein the plurality of apertures are uniformly spaced about the first portion.

8. The device of claim 1, wherein the sensor module retaining device is made of at least one substantially rigid material selected from the group consisting of titanium, stainless steel, MP35N alloy, plastic, and ceramic.

9. The device of any of claim 1, wherein at least a portion of the first portion is textured to facilitate adhesion of the sensor module retaining device to the outer surface of the blood vessel.

10. The device of any of claim 1, wherein the open channel is sized to receive a sensor module having one of:
a width equal to a width of the blood vessel; and
a width larger than a width of the blood vessel.

11. A system for measuring vital signs of a patient having a blood vessel, comprising:
a sensor module having a housing, the sensor module being configured to measure a blood vessel blood pressure waveform; and
a sensor module retaining device releasably coupled to the sensor module, the sensor module retaining device including:
a first portion defining an open loop sized and configured to contour around an outer surface of the blood vessel; and
a second portion and a third portion opposite the second portion, the second and third portions extending distally away from the open loop and defining an open channel therebetween, the second and third portions being contiguous with and converging inwards towards the first portion.

12. The system of claim 11, wherein the sensor module includes a diaphragm coupled to the housing and wherein the sensor module is received within the open channel such that the diaphragm is in contact with the outer surface of the blood vessel.

13. The system of claim 11, wherein at least the first portion of the sensor module retaining device defines a plurality of apertures to promote tissue ingrowth.

14. The system of claim 11, wherein the first and second portions each define at least one opening sized and configured to engage at least one corresponding mating member located on the sensor module.

15. The system of claim 11, wherein the second and third portions each have a proximal segment and a distal segment contiguous with the proximal segment, the proximal segment of the second and third portions being contiguous with the first portion.

16. The system of any claim 11, wherein the open loop is defined between the first and second ends.

17. The system of claim 11, wherein the first portion defines an open lumen and the open channel has a width larger than a width of the open lumen.

18. The system of claim 13, wherein the plurality of apertures are uniformly spaced about the first portion.

19. The system of claim 11, wherein at least a portion of the first portion is textured to facilitate adhesion of the sensor module retaining device to the exterior surface of the blood vessel.

20. A system for measuring vital signs of a patient having a blood vessel, comprising:
a sensor being configured to measure a blood vessel blood pressure waveform, the sensor including a sensor module having a housing and a diaphragm, the diaphragm being coupled to the housing;
a sensor module retaining device releasably coupled to the sensor module, the sensor module retaining device including:
a first portion defining an open loop sized and configured to contour around an outer surface of the blood vessel, the first portion of the sensor module retaining device further defining a plurality of apertures to promote tissue ingrowth; and
a second portion and a third portion opposite the second portion, the second and third portions extending distally away from the open loop and defining an open channel therebetween, the second and third portions being contiguous with and converging inwards towards the first portion, and when the sensor module is received within the housing the diaphragm is in contact with the outer surface of the blood vessel.
